(19) United States
(12) Patent Application Publication
(10) Pub. No.: US 2018/0311151 A1
(43) Pub. Date: Nov. 1, 2018

(54) AN EXPANDABLE PLUG BODY FOR USE IN VAGINA AND ITS APPLICATION

(71) Applicant: HARBIN OT PHARMACEUTICAL CO., LTD., Heilongjiang (CN)

(72) Inventor: Mingshi QIU, Heilongjiang (CN)

(73) Assignees: Mingshi Qui, Heilongjiang (CN); HARBIN OT PHARMACEUTIAL CO., LTD., Heilongjiang, (CN)

(21) Appl. No.: 15/538,295

(22) PCT Filed: May 11, 2017
(86) PCT No.: PCT/CN2017/083889

§ 371 Date: Jan. 25, 2018

(30) Foreign Application Priority Data
Apr. 27, 2017 (CN) 201710286482.5

(51) Int. Cl. A61K 9/00; A61F 13/20

(52) U.S. Cl. CPC A61K 9/0036; A61F 13/2002; A61F 13/2051

(57) ABSTRACT

The present invention relates to an expandable plug body for use in a vagina which is expandable at a predetermined value in an aqueous solution and has an expansion value of 1 to 30 at saturated water absorption. Effective components may be immersed in the interior of the expandable plug body, or dissolved and dispersed in the material wrapping the expandable plug body. The expandable plug body is primarily used in the vagina. The present invention provides an expandable plug body having some unique physical properties such as expanding at a predetermined expansion value, expanding at a specific pH value, and periodically expanding, so that it has a good performance when used as a medicine, a cosmetic, and a disinfection product. After the application of the original liquid of the expandable plug body onto the vagina for 3˜5 min, antibacterial rates of which on E. coli, Staphylococcus aureus and Candida albicans are all in the range of 89˜95%.

### TECHNICAL FIELD

The present disclosure relates to the technical field of hygienic products, and in particular to an expandable plug body for use in a vagina and the application thereof.

### BACKGROUND

At present, novel vectors with specific expansion properties is of great significance in the fields of foods, medicines, cosmetics, disinfection products, health products, health care supplies and medical devices, and a large number of valuable products can be derived therefrom. For example, an ordinary sterile sliver has a wide range of uses, which can be made into medical absorbent cottons commonly used in hospitals, disposable sanitary products, sanitary pads used during female menstrual period, and can also be made into built-in slivers for suppositories, and the like.

However, the requirements for each kind of products in the fields such as cosmetics, disinfection products, and medical devices are different; resulting in most of the vectors with specific physical and chemical properties can only be specifically applied to a certain field instead of all fields. At present, the expansion of the ordinary absorbent cotton is irregular, and its material is single, and it is impossible to flexibly select an appropriate product according to the specific application field.

Although the following patents have improved the physical properties and applications of the absorbent cotton from different aspects, for example CN1607930A discloses a tampon in particular for feminine hygiene, CN1034938A discloses an expansion cellulose sponge, CN1980619A discloses an intravaginal device with controlled expansion, CN101166493A discloses a dual mode absorbent tampon, and CN102164567A discloses a tampon expanding according to anthropometry. However, problems such as narrow application fields, single material, and disordered expansion of the “vectors with specific expansion properties” are still not fully resolved; therefore it has remarkable significance to get a novel expandable plug body for use in a vagina.

### SUMMARY

In view of the currently existing technical problems, one object of the present disclosure is to provide an expandable plug body for use in a vagina and the application thereof, the expandable plug body for use in a vagina as described in the present disclosure is expandable in an aqueous solution at a predetermined expansion value, having an expansion value of 1-30 when the absorption of water reaches saturation, wherein active ingredients can immerge into the interior of the expandable plug body, or dissolve and disperse in the materials wrapping the expandable plug body.

To achieve this object, the present disclosure adopts the following technical solutions:

In the first aspect, an expandable plug body for use in a vagina is provided in the present disclosure, wherein keels are provided on the surface of the expandable plug body in axial or radial direction, wherein 3-5 keels are provided in the radial direction and 8-10 keels are provided in the axial direction.

According to the present disclosure, the number of keels provided respectively on the surface of the expandable plug body for use in a vagina in axial or radial direction has an important effect on the expansion value of the body when the absorption of water reaches saturation. By arranging 3-5 keels in the radial direction and 8-10 keels in the axial direction, on the surface of the expandable plug body, it is possible to rapidly increase the expansion value of the expandable plug body when the absorption of water reaches saturation, to achieve rapid expansion, so that active ingredients can fully immerge into the interior of the expandable plug body, or fully dissolve and disperse in the materials wrapping the expandable plug body, thereby achieving better drug efficacy.

In the present disclosure, 3-5 keels, for example 3, 4 or 5, preferably 3-4 keels are provided in the radial direction; and 8-10 keels, for example 8, 9 or 10, preferably 9-10 keels are provided in the axial direction, of the expandable plug body.

For the keel arrangement of the expandable plug body according to the present disclosure, the use of more axially provided keels will contribute to the rapid expansion of the expandable plug body at a lower water absorption and allow active ingredients to enter the interior of the expandable plug body, so that it can more effectively exert the effect of the active ingredients.

It has been found by the inventor that when more keels are arranged axially, it makes possible to achieve rapid expansion of the expandable plug body, however, at most 10 keels can be arranged, once it exceeds 10 keels, the expansion of the vector will be difficult to control; when less keels are arranged radially, likewise it will contribute to the rapid expansion of the vector at a lower water absorption, however a minimum of 3 keels should be arranged, once it is less than 3 keels, uneven expansion of the expandable plug body will occur, such that the active ingredient fail to fully act on the subject, thereby affecting its efficacy.

According to the present disclosure, the keels arranged in the axial and radial directions on the surface of the expandable plug body have matching relationship and can interact with each other; when 3-4 keels are arranged in the radial direction and 9-10 keels are arranged in the axial direction, the expansion value of the expandable plug body is optimal when its absorption of water reaches saturation, so that the active ingredients can play the greatest role.

The term “active ingredient” as used in the present disclosure refers to a substance that can act on the human body and play a role of treatment, health care, disinfection, cosmetic, and cleansing, etc.

According to the present disclosure, the outer periphery of the expandable plug body is wrapped with a nonwoven fabric as a protective layer, and those skilled in the art will appreciate that the nonwoven fabric may generally refer to various common materials in the art, for example any one or a mixture of at least two of polyester fiber, polyolefin fiber, polyamide fiber, polyacrylonitrile fiber or regenerated fiber, wherein the typical but non-limiting mixture is a mixture of polyolefin fiber and polyamide fiber; a mixture of polyacrylonitrile fiber and various natural fibers, and a mixture of polyacrylonitrile fiber and regenerated fiber.

Specifically, the nonwoven fabric can be any one or a mixture of at least two selected from the group consisting of polypropylene fiber, polyester fiber, polyamide fiber, viscose fiber, acrylic fiber, polyethylene fiber or polyvinyl chloride fiber, wherein the typical but non-limiting combination is a mixture of polypropylene fiber and polyester fiber, a mixture of polyamide fiber and viscose fiber, and a mixture of acrylic fiber and polyethylene fiber.

In the present disclosure, the nonwoven fabric material wrapped on the outer periphery of the expandable plug body is associated with the expandable plug body itself, that is, when the nonwoven fabric is selected from some specific materials, it will have a crucial effect on the expansion value of the expandable plug body when the absorption of water reaches saturation.

It has been found by the inventor through studies that when the nonwoven fabric is selected from any one or a combination of at least two of polypropylene fiber, polyamide fiber, acrylic fiber or polyvinyl chloride fiber in the above materials, it can play a synergistic role with the expandable plug body, so that not only it can function as a protective layer, but it can make the optimal expansion value of the expandable plug body be rapidly achieved when the absorption of water reaches saturation.

The expandable plug body of the present disclosure may exist in the form of a tampon, or may be in the form of other plug bodies containing a nonwoven fabric material.

In the present disclosure, the expandable plug body comprises a high expansion zone and a low expansion zone; wherein the expansion value of the high expansion zone is 20-30, for example 20, 21, 22, 23, 24, 25, 26, 27, 28, 29 or 30; and the expansion value of the low expansion zone is 1-10, for example 1, 2, 3, 4, 5, 6, 7, 8, 9 or 10.

Preferably, the material for preparing the high expansion zone comprises at least cellulose fiber, cotton and viscose fiber; and the mass ratio of the cellulose fiber, cotton and viscose fiber is 7-10:1-2:1-3, for example 7:1:1, 8:2:1, 9:1:3, 10:2:1, 7:2:3, 8:1:3 or 10:2:3; and the material for preparing the low expansion zone comprises at least cellulose fiber, cotton and viscose fiber; and the mass ratio of the cellulose fiber, cotton and viscose fiber is 4-6:3-5:1-2, for example 4:3:1, 5:4:1, 6:5:2, 4:5:1, 6:3:1, 5:5:1 or 6:3:2.

Preferably, different parts of the expandable plug body have the same expansion value but different expanding orders, preferably the outer end of the expandable plug body firstly expands; alternatively, different parts of the expandable plug body have different expansion values, and it is preferable that the expansion value of the keel is larger than that of other parts.

The preferable sizes of the expandable plug body for use in a vagina according to the present disclosure in most fields are: a diameter of 3-15 mm, for example 3 mm, 5 mm, 6 mm, 8 mm, 10 mm, 11 mm, 12 mm, 13 mm or 15 mm; a weight deviation of 1-4 g, for example 1 g, 1.2 g, 2 g, 2.5 g, 3 g or 4 g; a length of 10-60 mm, for example 10 mm, 20 mm, 22 mm, 31 mm, 45 mm, 52 mm or 60 mm; and a pull wire length of ≥50 mm, for example 50 mm, 60 mm, 62 mm, 67 mm, 70 mm or 80 mm; preferably the expandable plug body has a diameter of 10-11 mm, a weight deviation of 2.1-2.4 g, a length of 43-46 mm, and a pull wire length of ≥100 mm.

The material for the expandable plug body for use in a vagina according to the present disclosure may be selected from the group consisting of cotton, fiber, resin, polyurethane sponge, cellulose fiber, paper, nonwoven fabric, polymer absorbent material, foam material, superabsorbent, and hydrogel; the cellulose fiber includes wood pulp, cotton, rayon, viscose fiber, and Lyocell fiber; the fiber includes synthetic fiber or natural fiber; the synthetic fiber includes polyolefin, polyester, polyamide, rayon, superabsorbent fiber, polyacrylonitrile-based fiber, viscose fiber, and regenerated cellulose fiber, etc; and the natural fiber includes wool, cotton, linen, hemp, jute, ramie, and wood pulp, etc.

The interior of the expandable plug body for use in a vagina according to the present disclosure is provided with a built-in expandable plug body which will expand after the expandable plug body exerts its expansive action.

Preferably, the built-in expandable plug body is 2 or more than 2, for example 2, 3, 4 or 5, etc. When there is a plurality of built-in expandable plug bodies, one built-in expandable plug body can be placed in another built-in expandable plug body, or a plurality of built-in expansion plug bodies can be placed in parallel in the same expandable plug body or built-in expandable plug body.

The weight ratio of the active ingredient to the expandable plug body of the present disclosure is preferably 0.1:50-50:1, when the built-in expandable plug body is contained, the weight ratio of the active ingredient to the sum of the expandable plug body and the built-in expandable plug body is 0.1:50-50:1.

The present disclosure further provides an application of the expandable plug body for use in a vagina as described in the first aspect.

The application of the expandable plug body for use in a vagina as described in the present disclosure is illustrated by way of examples below.

The first application is the use of the expandable plug body as a disinfection product which may be a bacteriostatic maintenance agent, wherein its effective components comprise at least one active ingredient having cleaning effect on the vagina internal environment and at least one macromolecular excipient. The active ingredient includes, but is not limited to, chlorhexidine acetate or chitosan, etc. The macromolecular excipient is algal polysaccharide and/or liquid paraffin. The preferred application is that the parts by weight of chlorhexidine acetate and chitosan are both 100.

The second application is the use of the expandable plug body as a cosmetic which is a cleaning product with a cleaning effect on the vagina made by adding the expandable plug body into a cleaning agent. The cleaning product can also include a booster made from medicinal polyethylene or polypropylene as the material, it is required that the booster has uniform color without obvious color aberration, and smooth and flat surface without deformation, obvious scratches, sand hole and oil stain.

Preferably, the cleaning agent comprises the following raw materials in parts by weight: 30-60 parts of liquid paraffin, 10-40 parts of lanolin, 10-40 parts of olive oil, 0.01-10 parts of carbomer, and 0.01-10 parts of hyaluronic acid, preferably comprising the following raw materials in parts by weight: 44 parts of liquid paraffin, 27 parts of lanolin, 28 parts of olive oil, 0.5 part of carbomer, and 0.5 part of hyaluronic acid.

Compared with the prior art, the present disclosure has at least the following beneficial effects:

According to the present disclosure, by arranging 3-5 keels in the radial direction, and 8-10 keels in the axial direction, on the surface of the expandable plug body, it is possible to rapidly increase the expansion value of the expandable plug body when the absorption of water reaches saturation, to achieve rapid expansion, so that active ingredients can fully immerge into the interior of the expandable plug body, or fully dissolve and disperse in the materials wrapping the expandable plug body, thereby achieving better drug efficacy. An antibacterial rate of 89%-95% against Escherichia coli, Staphylococcus aureus and Candida albicans can be achieved only after the stock solution acts for 3-5 minutes.

### BRIEF DESCRIPTION OF THE DRAWINGS

FIG. 1 is a schematic diagram of the structure of the expandable plug body for use in a vagina according to the present disclosure, wherein 1 represents radial keel and 2 represents axial keel.

The present disclosure will be further described in detail below. However, the following examples are merely illustrative examples of the present disclosure, which do not represent or limit the claimed scope of the present disclosure that is only defined by the claims.

### DETAIL DESCRIPTION

The technical solution of the present disclosure will be further described below by way of specific embodiments in combination with accompanying drawing.

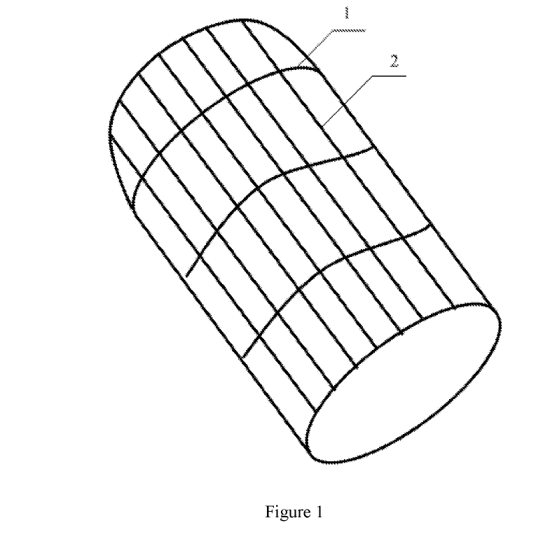

FIG. 1 shows the expandable plug body for use in a vagina according to the present disclosure which is provided with a keel 1 in the radial direction, and a keel 2 in the axial direction, on the surface of the body.

In order to better illustrate the present disclosure, and easily understand the technical solution of the present disclosure, the typical but non-limiting examples of the present disclosure are as follows:

### Example 1 Bacteriostatic Maintenance Agent

The expandable plug body for use in a vagina of this example was made of cellulose fiber, jute and viscose fiber as materials in a ratio of 5.5:2:1, wherein the cellulose fiber was supplied by Shanghai Royang Innovative Material Technologies Co., Ltd. 3 keels were arranged in the radial direction, and 8 keels were arranged in the axial direction, on the surface of the body, the pull-out end firstly expanded, the expansion value of the pull-out end and the incoming end were the same; one end of the expandable plug body was provided with a cotton string, wherein a nonwoven fabric material of polypropylene fiber was used as the protective layer.

The active ingredients carried by the expandable plug body were chlorhexidine acetate and chitosan in a weight ratio of 100:100, and fillers of algal polysaccharide and liquid paraffin.

In this Example, the antibacterial rates against Escherichia coli, Staphylococcus aureus and Candida albicans were all 92% upon detection after the stock solution carried by the bacteriostatic maintenance agent acted in the vagina for 3 minutes.

### Example 2 Bacteriostatic Maintenance Agent

Compared with Example 1, Example 2 was the same as Example 1 except that 4 keels were arranged in the radial direction, and 9 keels were arranged in the axial direction, on the surface of the body.

In this Example, the antibacterial rates against Escherichia coli, Staphylococcus aureus and Candida albicans were 94%, 92.4% and 92.5%, respectively after the stock solution carried by the bacteriostatic maintenance agent acted in the vagina for 3 minutes.

### Example 3 Bacteriostatic Maintenance Agent

Compared with Example 1, Example 3 was the same as Example 1 except that 3 keels were arranged in the radial direction, and 9 keels were arranged in the axial direction, on the surface of the body.

In this Example, the antibacterial rates against Escherichia coli, Staphylococcus aureus and Candida albicans were all 93% after the stock solution carried by the bacteriostatic maintenance agent acted in the vagina for 3 minutes.

### Example 4 Bacteriostatic Maintenance Agent

Compared with Example 1, Example 4 was the same as Example 1 except that 3 keels were arranged in the radial direction, and 10 keels were arranged in the axial direction, on the surface of the body.

In this Example, the antibacterial rates against Escherichia coli, Staphylococcus aureus and Candida albicans were 94.8%, 94.9% and 95%, respectively after the stock solution carried by the bacteriostatic maintenance agent acted in the vagina for 3 minutes.

### Comparative Example 1 Bacteriostatic Maintenance Agent

Compared with Example 1, Comparative Example 1 was the same as Example 1 except that 8 keels were arranged in the radial direction, and 3 keels were arranged in the axial direction, on the surface of the body.

In this Comparative Example, the antibacterial rates against Escherichia coli, Staphylococcus aureus and Candida albicans were 65%, 58% and 62%, respectively after the stock solution carried by the bacteriostatic maintenance agent acted in the vagina for 3 minutes.

By comparison between Example 1 and Comparative Example 1, it can be concluded that when more keels are arranged axially and relatively less keels are arranged radially, it can help the active ingredients to function better, so that the antibacterial rates against Escherichia coli, Staphylococcus aureus and Candida albicans can be greatly increased.

### Comparative Example 2 Bacteriostatic Maintenance Agent

Compared with Example 1, Comparative Example 2 was the same as Example 1 except that 1 keel was arranged in the radial direction, and 8 keels were arranged in the axial direction, on the surface of the body.

In this Comparative Example, the antibacterial rates against Escherichia coli, Staphylococcus aureus and Candida albicans were 67.5%, 60% and 68%, respectively after the stock solution carried by the bacteriostatic maintenance agent acted in the vagina for 3 minutes.

By comparison between Example 1 and Comparative Example 2, it can be concluded that when the same number of keels is arranged in the axial direction, but the number of keels in the radial direction is reduced, it will delay the expansion of the expandable plug body to a certain extent, resulting in uneven expansion, so that the active ingredients cannot fully act on the subject, thereby reducing its efficacy.

### Comparative Example 3 Bacteriostatic Maintenance Agent

Compared with Example 1, Comparative Example 3 was the same as Example 1 except that 3 keels were arranged in the radial direction, and 5 keels were arranged in the axial direction, on the surface of the body.

In this Comparative Example, the antibacterial rates against Escherichia coli, Staphylococcus aureus and Candida albicans were 67%, 67.8% and 70%, respectively after the stock solution carried by the bacteriostatic maintenance agent acted in the vagina for 3 minutes.

By comparison between Example 1 and Comparative Example 3, it can be concluded that when the same number of keels are arranged in the radial direction, but the number of keels in the axial direction is reduced, likewise it will delay the expansion of the expandable plug body to a certain extent, resulting in uneven expansion, so that the active ingredients cannot fully act on the subject, thereby reducing its efficacy.

### Comparative Example 4 Bacteriostatic Maintenance Agent

Compared with Example 1, Comparative Example 4 was the same as Example 1 except that 6 keels were arranged in the radial direction, and 12 keels were arranged in the axial direction, on the surface of the body.

In this Comparative Example, the antibacterial rates against Escherichia coli, Staphylococcus aureus and Candida albicans were 70%, 65.1% and 68.4%, respectively after the stock solution carried by the bacteriostatic maintenance agent acted in the vagina for 3 minutes.

By comparison between Example 1 and Comparative Example 4, it can be concluded that when more keels are arranged radially and axially, it will make the expansion degree of the vector to be difficult to control, resulting in uneven expansion, likewise it will reduce the efficacy of the active ingredients.

### Example 5 Bacteriostatic Maintenance Agent

Compared with Example 1, Example 5 was the same as Example 1 except that the polyamide fiber was used as the nonwoven fabric material.

In this Example, the antibacterial rates against Escherichia coli, Staphylococcus aureus and Candida albicans were 89%, 92% and 94%, respectively after the stock solution carried by the bacteriostatic maintenance agent acted in the vagina for 4 minutes.

### Example 6 Bacteriostatic Maintenance Agent

Compared with Example 1, Example 6 was the same as Example 1 except that the acrylic fiber was used as the nonwoven fabric material.

The antibacterial rates against Escherichia coli, Staphylococcus aureus and Candida albicans were 92%, 93.1% and 94.2%, respectively after the stock solution carried by the bacteriostatic maintenance agent acted in the vagina for 5 minutes.

### Example 7 Cleaning Product

On the surface of the expandable plug body for use in a vagina of this Example, 3 keels were arranged in the axial direction, and 10 keels were arranged in the radial direction, and the segment contained keels were the high expansion zone, the remainders were the low expansion zone. The materials for preparing the high expansion zone of the expandable plug body comprised cellulose fiber, cotton and viscose fiber, and the ratio of the cellulose fiber, cotton and viscose fiber was 7:1:1; and the materials for preparing the low expansion zone of the expandable plug body comprised cellulose fiber, cotton and viscose fiber, and the ratio of the cellulose fiber, cotton and viscose fiber was 4:3:1. The cellulose fiber was supplied by Shanghai Royang Innovative Material Technologies Co., Ltd. The pull-out end and the incoming end simultaneously expanded, but the expansion values of the both were different.

In this Example, the active ingredients carried by the expandable plug body were 44% of liquid paraffin, 27% of lanolin, 28% of olive oil, 0.5% of carbomer and 0.5% of hyaluronic acid.

The antibacterial rates against Escherichia coli, Staphylococcus aureus and Candida albicans were 92.5%, 92.8% and 95%, respectively after the stock solution carried by the cleaning product acted in the vagina for 3 minutes.

### Example 8 Cleaning Product

On the surface of the expandable plug body for use in a vagina of this Example, 3 keels were arranged in the axial direction, and 10 keels were arranged in the radial direction, and the segment contained keels were the high expansion zone, the remainders were the low expansion zone. The materials for preparing the high expansion zone of the expandable plug body comprised cellulose fiber, cotton and viscose fiber, and the ratio of the cellulose fiber, cotton and viscose fiber was 8:2:1; and the materials for preparing the low expansion zone of the expandable plug body comprised cellulose fiber, cotton and viscose fiber, and the ratio of the cellulose fiber, cotton and viscose fiber was 5:5:2. The cellulose fiber was supplied by Shanghai Royang Innovative Material Technologies Co., Ltd. The pull-out end and the incoming end simultaneously expanded, but the expansion values of the both were different.

In this Example, the active ingredients carried by the expandable plug body were 30% of liquid paraffin, 38% of lanolin, 15% of olive oil, 10% of carbomer and 7% of hyaluronic acid.

The antibacterial rates against Escherichia coli, Staphylococcus aureus and Candida albicans were 91.0%, 90.8% and 92%, respectively after the stock solution carried by the cleaning product acted in the vagina for 3 minutes.

In conclusion, an expandable plug body for use in a vagina is provided in the present disclosure, the active ingredients of which can immerge into the interior of the expandable plug body, or dissolve and disperse in the materials wrapping the expandable plug body. The antibacterial rates against Escherichia coli, Staphylococcus aureus and Candida albicans are between 89% and 95% after the stock solution in the expandable plug body acts on the vagina for 3-5 minutes; therefore it has important application value.

Applicant has stated that although the detailed structural features of the present disclosure have been described by the above embodiments in the present disclosure, the present disclosure is not limited thereto, that is to say, it is not meant that the present disclosure has to be implemented depending on the above detailed structural features. It will be apparent to those skilled in the art that any improvements to the present disclosure, equivalent replacements to the selected components in the present disclosure, additions of ancillary components and choices of specific embodiments etc., all fall within the protection scope and the disclosure scope of the present disclosure.

While the preferred embodiments of the present disclosure are described in detail hereinbefore, the present disclosure is not limited to the specific details in the above embodiments, various simple variations of the technical solution of the present disclosure can be made within the scope of the technical concept of the present disclosure, and these simple variations fall within the protection scope of the present disclosure.

Additionally, it is to be noted that the specific technical features described in the above specific embodiments can be combined in any suitable manner without contradiction with the present disclosure, and in order to avoid unnecessary repetition, various possible combinations will not be described in the present disclosure.

In addition, any combination of the various different embodiments of the present disclosure may be made as long as it does not deviate from the spirit of the present disclosure and should likewise be regarded as the disclosure of the present invention.

1.-10. (canceled)

11. An expandable plug body for use in a vagina, wherein keels are provided on the surface of the expandable plug body in axial or radial direction, wherein 3-5 keels are provided in the radial direction and 8-10 keels are provided in the axial direction.

12. The expandable plug body for use in a vagina of claim 11, wherein the outer periphery of the expandable plug body is wrapped with a non-woven fabric as a protective layer; the nonwoven fabric is any one or a mixture of at least two selected from the group consisting of polyester fiber, polyolefin fiber, polyamide fiber, polyacrylonitrile fiber or regenerated fiber.

13. The expandable plug body for use in a vagina of claim 12, wherein the nonwoven fabric is any one or a mixture of at least two of polypropylene fiber, polyester fiber, polyamide fiber, viscose fiber, acrylic fiber, polyethylene fiber or polyvinyl chloride fiber.

14. The expandable plug body for use in a vagina of claim 11, wherein the expandable plug body comprises a high expansion zone and a low expansion zone.

15. The expandable plug body for use in a vagina of claim 14, wherein the expansion value of the high expansion zone is 20-30; and the expansion value of the low expansion zone is 1-10.

16. The expandable plug body for use in a vagina of claim 14, wherein the material for preparing the high expansion zone comprises at least cellulose fiber, cotton and viscose fiber; and the mass ratio of the cellulose fiber, cotton and viscose fiber is 7-10:1-2:1-3.

17. The expandable plug body for use in a vagina of claim 14, wherein the material for preparing the low expansion zone comprises at least cellulose fiber, cotton and viscose fiber; and the mass ratio of the cellulose fiber, cotton and viscose fiber is 4-6:3-5:1-2.

18. The expandable plug body for use in a vagina of claim 11, wherein different parts of the expandable plug body have same expansion value but different expanding orders.

19. The expandable plug body for use in a vagina of claim 18, wherein the outer end of the expandable plug body firstly expands.

20. The expandable plug body for use in a vagina of claim 11, wherein different parts of the expandable plug body have different expansion values.

21. The expandable plug body for use in a vagina of claim 20, wherein the expansion value of the keel is larger than that of the other parts.

22. The expandable plug body for use in a vagina of claim 11, wherein the expandable plug body has a diameter of 3-15 mm, a weight deviation of 1 to 4 g, a length of 10-60 mm, and a pull wire length of ≥50 mm.

23. The expandable plug body for use in a vagina of claim 22, wherein the expandable plug body has a diameter of 10-11 mm, a weight deviation of 2.1-2.4 g, a length of 43-46 mm, and a pull wire length of ≥100 mm.

24. The expandable plug body for use in a vagina of claim 11, wherein the interior of the expandable plug body is provided with a built-in expandable plug body which will expand after the expandable plug body exerts its expansive action.

25. The expandable plug body for use in a vagina of claim 24, wherein the built-in expandable plug body is at least 2.

26. The expandable plug body for use in a vagina of claim 25, wherein the built-in expandable plug body is 3-5.

27. A product containing the expandable plug body for use in a vagina of claim 11, wherein the product is a drug, a cosmetic, or a disinfection product.

28. The product of claim 27, wherein the expandable plug body is used in a cosmetic.

29. The product of claim 28, wherein the cosmetic is made by immersing the expandable plug body in a cleaning agent;
the cleaning agent comprises the following raw materials in parts by weight: 30-60 parts of liquid paraffin, 10-40 parts of lanolin, 10-40 parts of olive oil, 0.01-10 parts of carbomer, and 0.01-10 parts of hyaluronic acid.

30. The product of claim 29, wherein the cleaning agent comprises the following raw materials in parts by weight: 44 parts of liquid paraffin, 27 parts of lanolin, 28 parts of olive oil, 0.5 part of carbomer, and 0.5 part of hyaluronic acid.
